(19) United States
(12) Patent Application Publication
(10) Pub. No.: US 2020/0009324 A1
(43) Pub. Date: Jan. 9, 2020

(54) SYSTEMS AND METHODS FOR TRIGGERING A DRUG INJECTION DEVICE

(71) Applicant: Verily Life Sciences LLC, South San Francisco, CA (US)

(72) Inventors: Daniel Barrows, Bloomfield, NY (US); Grant Smith, Belmont, CA (US)

(73) Assignee: Verily Life Sciences LLC, South San Francisco, CA (US)

(21) Appl. No.: 16/449,922

(22) Filed: Jun. 24, 2019

Related U.S. Application Data
(60) Provisional application No. 62/695,577, filed on Jul. 9, 2018.

(51) Int. Cl. A61M 5/20; A61M 5/19

(52) U.S. Cl. CPC A61M 5/2046; A61M 5/172; A61M 2005/206; A61M 5/19

(57) ABSTRACT

Systems and methods for triggering a drug injection device are disclosed. One disclosed system for injecting a substance into a patient includes: a chamber comprising a propellant; a light source mechanically coupled to the chamber, wherein energy from the light source ignites the propellant; and a power source electrically coupled to the light source via a control circuit, wherein the control circuit applies power to activate the light source.

### CROSS REFERENCE To RELATED APPLICATION

This application claims the benefit of U.S. application Ser. No. 62/695,577, filed Jul. 9, 2018, titled “Systems And Methods For Triggering A Drug Injection Device,” which is incorporated herein by reference in its entirety.

### FIELD

The present application generally relates to triggering systems for drug injection devices, and more specifically relates to systems and methods for an optically triggered drug injection device.

### BACKGROUND

People with certain medical conditions may require doses of medication in response to certain physiological conditions. For example, a diabetic may monitor her blood sugar and, if it gets too high, inject insulin to help lower the blood sugar levels. Conversely, she may eat some food if her blood sugar gets too low. Another example is a person with an allergy to peanuts or insect stings that experiences anaphylaxis as a result of contact with the allergen. To respond to the anaphylaxis, the person may inject herself with epinephrine, such as with an off-the-shelf epinephrine injector, e.g., an EpiPen®.

### SUMMARY

Various examples are described for systems and methods for triggering wearable emergency drug injection devices. For example, one disclosed device for triggering a drug injection device comprises: a chamber comprising a propellant; a light source mechanically coupled to the chamber, wherein energy from the light source ignites the propellant; and a power source electrically coupled to the light source via a control circuit, wherein the control circuit applies power to activate the light source.

One disclosed example method for triggering a drug injection device comprises: receiving a control signal; controlling a power supply to apply power to a light source mechanically coupled to a chamber, wherein energy from the light source ignites a propellant within the chamber; and applying pressure to a piston to inject a substance into a patient.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1A shows an example wearable emergency drug injection device according to the present disclosure.

FIG. 1B shows another example wearable emergency drug injection device according to the present disclosure.

FIG. 1C shows another example wearable emergency drug injection device according to the present disclosure.

FIG. 1D shows another example wearable emergency drug injection device according to the present disclosure.

FIG. 1E shows another example wearable emergency drug injection device according to the present disclosure.

FIG. 2A shows an embodiment of an example system for triggering a drug injection device according to the present disclosure.

FIG. 2B shows another embodiment of an example system for triggering a drug injection device according to the present disclosure.

FIG. 2C shows another embodiment of an example system for triggering a drug injection device according to the present disclosure.

FIG. 3A shows an embodiment of an example system for triggering a blood extraction device according to the present disclosure.

FIG. 3B shows another embodiment of an example system for triggering a blood extraction device according to the present disclosure.

FIG. 3C shows another embodiment of an example system for triggering a blood extraction device according to the present disclosure.

FIG. 4 shows another embodiment of an example system for triggering a drug injection device according to the present disclosure.

FIG. 5 shows a flow chart for a method for triggering a drug injection device according to the present disclosure.

### DETAILED DESCRIPTION

Those of ordinary skill in the art will realize that the following description is illustrative only and is not intended to be in any way limiting. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

Illustrative Embodiment of Triggering a Drug Injection Device

A person with a medical condition, such as diabetes or a severe allergy to a substance, may use a wearable emergency drug injection device according to this disclosure. In this example, the person (also the “wearer”) obtains the device. The device has components to store and deliver a dose of an injectable substance, e.g., 1 milligram (“mg”) of glucagon powder and 1 milliliter (“ml”) of an activation solution that when mixed with the glucagon, activates the glucagon to enable it to be metabolized by the wearer.

In the illustrative embodiment, the injection device comprises a chamber with an ignitable propellant (e.g., nitrocellulose). One end of the chamber comprises a piston. Ignition of the propellant releases gasses that increase the pressure in the chamber and force the piston forward. When the piston moves forward it applies pressure to the injectable substance to move it forward. This pressure causes the injectable substance to travel through a hollow needle to be injected into the wearer (e.g., under the surface of the wearer's skin or into the wearer's bloodstream). Alternatively, in some embodiments, the piston may be configured to press the hollow needle forward into a wearer's skin. In such an embodiment, a second propellant and piston may apply pressure to cause the injectable substance to travel through the hollow needle. Alternatively, in still other embodiments, the needle may be used to extract blood from the wearer. In such an embodiment, an extraction mechanism may generate negative pressure within the chamber to extract blood from the wearer via the needle.

In the illustrative embodiment, the end of the chamber opposite the piston comprises a light source (e.g., a light emitting diode (LED), laser LED, or lamp). Light energy output by the light source is configured to ignite the propellant. For example, in the illustrative embodiment, when the light source is activated, the light source provides light energy to the propellant sufficient to create heat that causes the propellant to ignite. In the illustrative embodiment the light source is controlled by a control circuit (e.g., a processor), which controls power flow to the light source.

In the illustrative embodiment, the control circuit is coupled to a wireless receiver (e.g., a Bluetooth, Wi-Fi, or Near Field Communication (NFC) receiver). The receiver is communicatively coupled to a wearable sensor configured to monitor a condition of the wearer (e.g., a continuous glucose meter). In the illustrative embodiment, the wearable sensor is configured to trigger the injection device by igniting the propellant when a condition of the wearer exceeds a threshold. For example, in one embodiment, a continuous glucose monitor may trigger the injection device to provide insulin to the wearer upon detecting that the wearer's blood sugar has gone below a certain threshold. In another embodiment, a sensor may trigger an injection of epinephrine upon detecting that the wearer has come in contact with an allergen (e.g., nuts such as peanuts, shellfish, insects, e.g., stinging insects, animal dander, dust, or pollen).

This illustrative example is given to introduce the reader to the general subject matter discussed herein and the disclosure is not limited to this example. The following sections describe various additional non-limiting examples and examples of systems and methods for wearable emergency drug injection devices.

Illustrative System for Triggering a Drug Injection Device

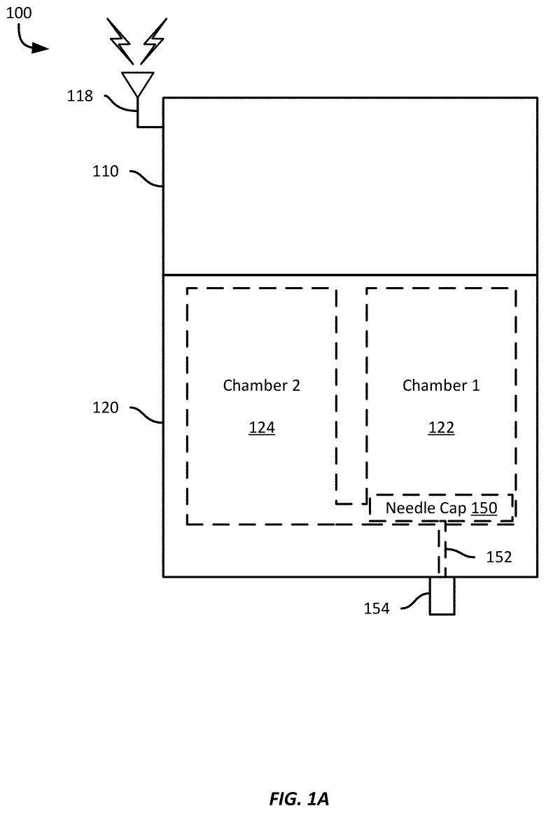

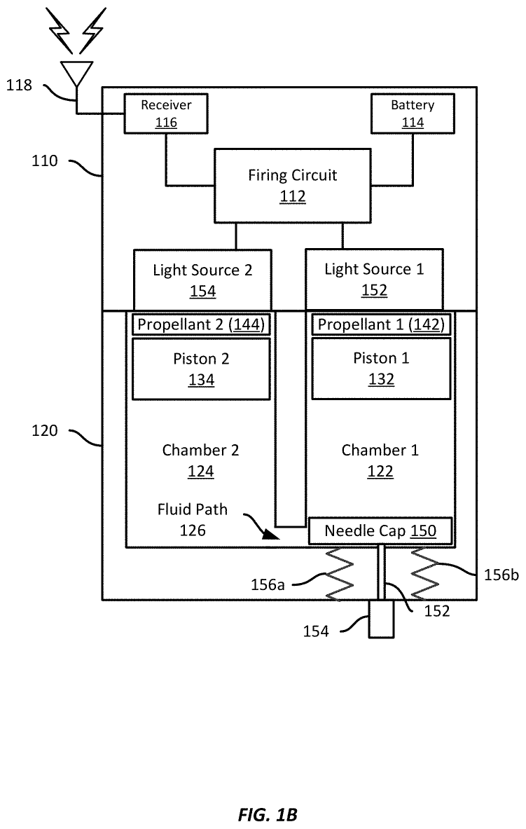

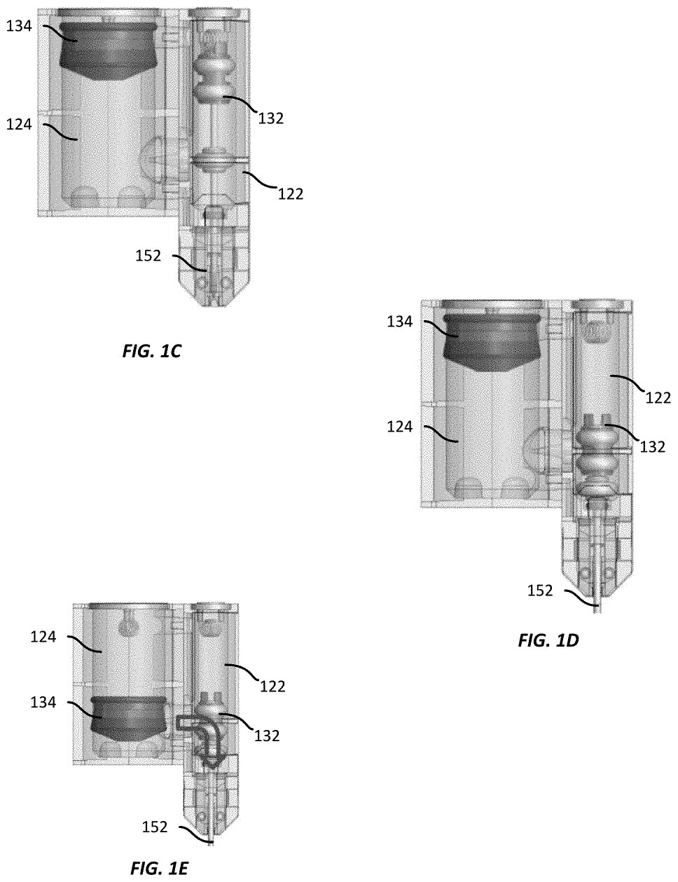

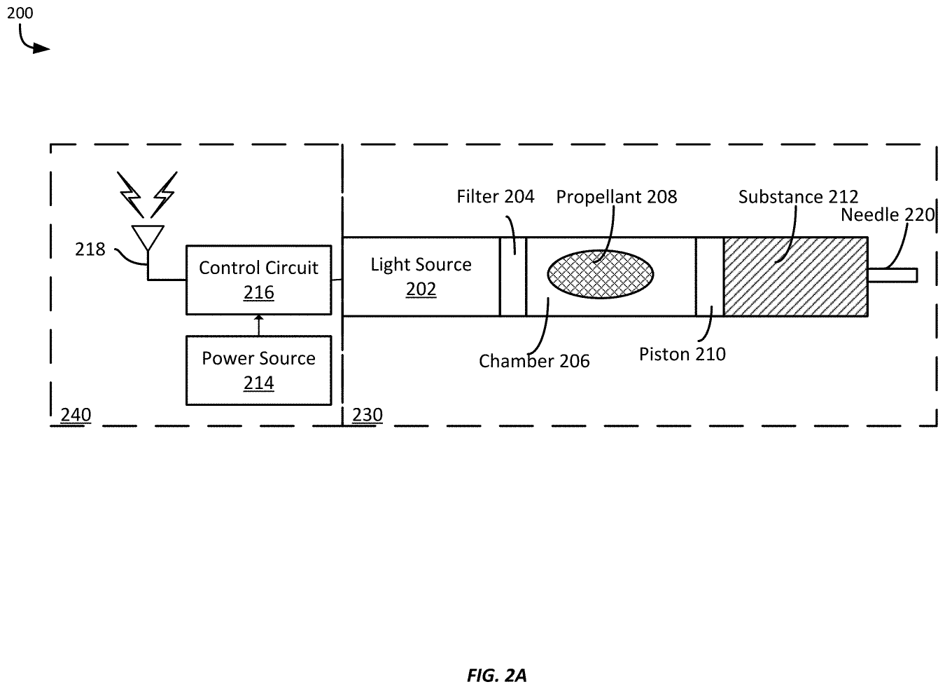

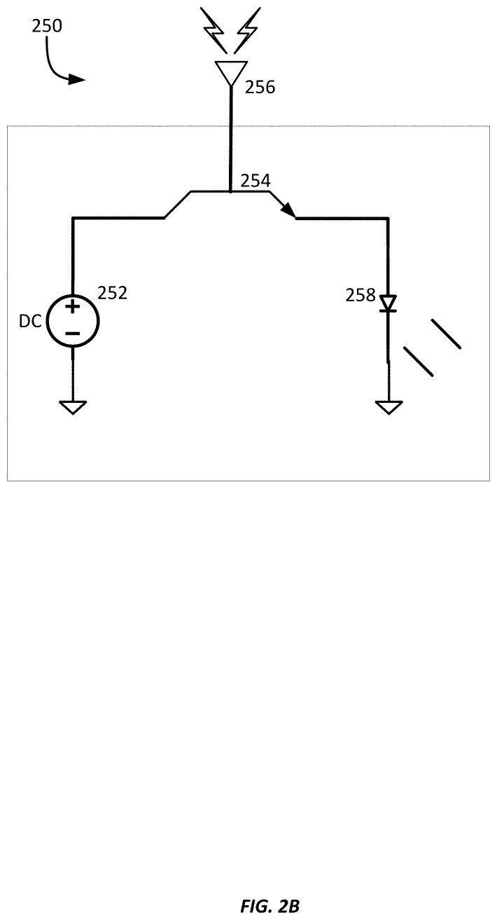

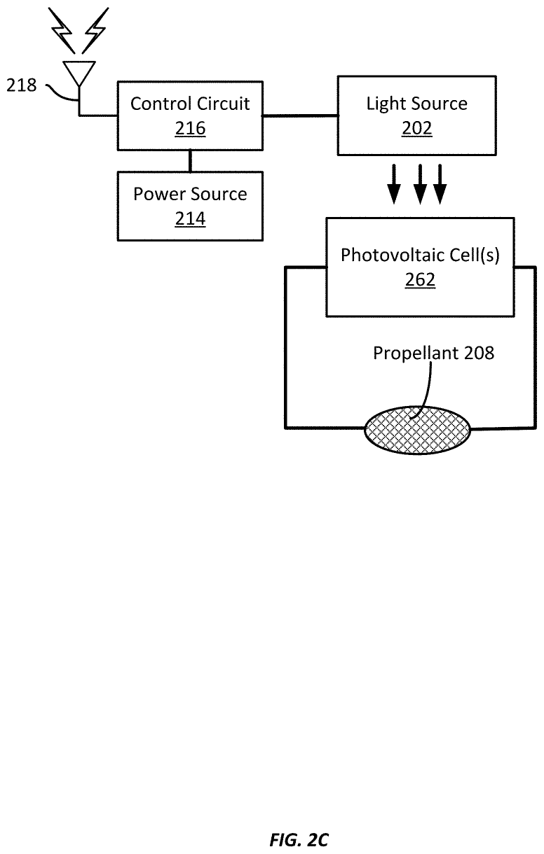

Turning now to FIG. 1A, FIG. 1A shows an example wearable emergency drug injection device 100. As can be seen in FIG. 1A, the example device 100 has two portions 110, 120 that are connected, but are separable from each other. The first portion 110 has electronic components within it, which are described in more detail with respect to FIGS. 1B, 2A, and 2B, and an antenna 118 to receive wireless signals. The first portion 110 in this example is separable from the second portion 120 to allow for re-use of the electronics, while the second portion can be discarded after it has been used.

The second portion 120 has two chambers that can be used to store injectable material(s), as well as a hollow needle 152 and a needle cap 150 that can be used to drive the needle 152 through the needle guide 154 and into a person's skin. In this example, because the needle 152 is hollow, injectable material(s) can be forced out of one or both chambers, through the needle, and into the wearer.

The example device shown in FIG. 1A is designed to be worn flush against a wearer's body, such as on an upper arm or torso. The needle 152, as shown in FIG. 1A, is oriented to extend parallel to the wearer's skin; however, the needle guide 154 defines a curved path that forces the needle 152 to bend toward the wearer's skin at an angle departing from its initial orientation by approximately 30 degrees in this example. Thus, the needle 150, in this example, is formed of flexible materials, such as a nickel-titanium alloy (e.g., Nitinol), to allow the needle 152 to bend at angles of up to 30 degrees (or more) without breaking or obstructing the fluid path through the interior of the needle 152. In addition, the needle 152 in this example is a 22-gauge needle. Such a needle size may provide a diameter suitable for injecting fluid into the wearer while having a diameter that causes a tolerable amount of discomfort; however, other suitable needle diameters may be employed.

With respect to description of length, width, and height, the height of the device 100 shown in FIG. 1A refers to how far the device extends above the wearer's skin when worn as described above. The length and width, by contrast, refer to the dimensions of the perimeter of the device 100 shown in FIG. 1A.

Turning now to FIG. 1B, FIG. 1B shows a more detailed view of the interior of the first and second portions 110, 120 of the device 100. As discussed above, the second portion 120 defines two chambers 122, 124. Within each chamber 122, 124 is a piston 132, 134 which are initially positioned at one end of the respective chamber opposite an opening. Thus, when the pistons 132, 134 move, the contents of the corresponding chamber 122, 124 are expelled.

The pistons 132, 134 are sized to have approximately the same cross- sectional area as the corresponding chamber 122, 124 to prevent the contents of the chamber 122, 124 from sliding around the piston or, as will be described, gas pressure generated behind the piston from being dissipated by escaping around the piston 132, 134. In addition, in some examples, one or more of the pistons 132, 134 may have a ring seal attached around the perimeter of the piston 132, 134 to prevent such leakage of material or gasses past the piston 132, 134.

A propellant 142, 144 is disposed behind each piston 132, 134. When one of the propellants 142, 144 is activated, it generates pressure within the portion of the chamber behind the piston 132, 134, thereby forcing the piston toward the opposite end of the chamber.

In this example, each propellant 142, 144 comprises a nitrocellulose material, and propellant 1 (142) has a faster-burning nitrocellulose material than propellant 2 (144). For example, propellant 1 (142) in this example is a nitrocellulose in a cotton-based format, while propellant 2 (144) in this example is a nitrocellulose in a paper-based format. Selection of an appropriate propellant may be made based on the contents of the chamber.

For example, chamber 1 may have no injectable material in it, or may have an amount of an injectable powder, and thus may provide a mechanism for forcing the needle cap 150 and needle 152 downwards, thereby injecting the needle into the wearer's skin. In such an example, a faster-burning propellant may be used as concerns about over-pressurizing the chamber 122 may be reduced. In contrast, in this example, chamber 2 has an injectable fluid. Thus, a slower-burning or slower-acting propellant may be desired to allow time for the fluid to be expelled from the chamber 122 without over-pressurizing the chamber walls. In addition, selection of propellants may be made based on a desired firing sequence, a time to deliver a full dose of material to the wearer, or a time between insertion and retraction of the needle 152.

To enable the injectable material to move from the chamber(s) into the wearer, as discussed above, the needle 152 is hollow. In addition, a fluid path 126 is defined between the two chambers to allow injectable material to move from chamber 2 (124) through the fluid path 126 over the needle cap 150 and into the needle 150. And while it is referred to as a “fluid” path 126, it can allow solid (e.g., powders) or gaseous materials to flow as well. In addition, piston 1 (132) also defines a void that, after piston 1 (132) has been driven to the opposite end of the chamber 122, the void is exposed to the fluid path as well as the hollow portion of the needle. Thus, the combination of the fluid path 126, the void within piston 1 (132), and the hollow needle 152 provide a path for an injectable material to be expelled from the chamber(s) 122, 124 and into the wearer.

In addition, in this example, a pair of springs 156a-b is coupled to the needle cap to enable retraction of the needle 152. Thus, after the injectable substance has been expelled out of the chamber(s) and in to the wearer, the device 100 may retract the needle 152, via the springs 156a-b in this example. For example, the pressure generated by propellant 1 (142) may initially overcome the spring force, but as the pressure dissipates, e.g., via an exhaust port, the springs 156a-b may ultimately overcome the pressure and retract the needle 152. In other examples, other needle retraction mechanisms may be employed, such as another propellant charge located beneath the needle cap.

Further, in some embodiments, springs 156a-b (or another extraction mechanism), may be configured to generate negative pressure in Chamber 1 (122). In such an embodiment, rather than injecting a substance into the wearer, the needle 152 may instead be used to extract blood from the wearer. In such an embodiment, rather than injecting a substance from Chamber 1 (122) into the wearer, the chamber will instead be filled with blood extracted from the wearer. Such an embodiment may be useful for monitoring levels of substances in wearer's blood, e.g., blood glucose or blood alcohol monitoring.

While the second portion 120 includes the injectable material(s) and the mechanisms for inserting the needle 152 into the wearer and for storing and expelling the injectable material(s), the first portion 110 includes components to receive a command (or commands) to activate the propellant and inject the injectable material(s). In this example, the first portion 110 includes a firing circuit 112, a battery 114 or other electrical power source or connection, a wireless receiver 116, and an antenna 118. To activate the propellants 142, 144 and inject the injectable material into the wearer, in this example, a command is received via the antenna 118 and the receiver 116 from a remote device, such as the wearer's smartphone or a biosensor (e.g., a CGM), and is provided to the firing circuit 112. In response to receiving the command, the firing circuit 112 activates the propellants 142, 144 using power supplied by the battery 114.

In this example, the propellants 142, 144 are activated by optical energy output by light sources 152 and 154, as described in further detail below with regard to FIGS. 2A and 2B. The light sources 152 and 154 comprise any type of light source, e.g., an LED, and are controlled by firing circuit 112.

In addition to the firing circuit 112, other electronic components may be provided within the first portion 110 as well, such as battery charging circuitry, power and filtering circuitry, and a microcontroller, e.g., an ASIC defined on a field-programmable gate array (“FPGA”). Still further electronic components may be included within the first portion 110 to enable various features according to this disclosure.

While this example employs a wireless command to activate the firing circuit 112, in some examples, the device 100 may instead have a wired connection to another device, e.g., a biosensor, or may have a button or other wearer manipulatable device (“manipulandum”) to activate the firing circuit 112.

FIGS. 1C, 1D, and 1E show additional examples of wearable emergency drug injection device according to the present disclosure. Each of FIGS. 1C, 1D, and 1E show the same device at various operational stages according to the present disclosure. The device shown in these figures includes first and second pistons 132 and 134, first and second chambers 122 and 124, and needle 152. Each of these components is described in detail with regard to FIG. 1B.

The embodiment shown in FIG. 1C comprises an image of a device according to the embodiments described with regard to FIGS. 1A and 1B, prior to ignition of either propellant.

The embodiment shown in FIG. 1D comprises an image of a device according to the embodiments described with regard to FIGS. 1A and 1B, after ignition of the first propellant, but prior to ignition of second propellant, such that the needle 152 has been pushed forward, but no medication has been pushed forward by piston 134.

The embodiment shown in FIG. 1E comprises an image of a device according to the embodiments described with regard to FIGS. 1A and 1B, after ignition of both propellants, such that the needle 152 has been pushed forward and medication has been pushed by piston 134 from chamber 124 through needle 152.

Turning now to FIG. 2A, FIG. 2A shows an embodiment of an example system 200 for triggering a drug injection device. As shown in FIG. 2A the triggering system comprises injection system 230 and triggering circuitry 240. Injection system 230 comprises light source 202, filter 204, chamber 206, propellant 208, piston 210, injectable substance 212, and hollow needle 220.

The light source 202 comprises a device configured to output light energy upon receiving electrical current. For example, light source 202 may comprise one or more of a lamp or any type of LED (e.g., a white, blue, green, red, laser LED, or infrared LED). In some embodiment, the section of light source 202 facing chamber 206 may comprise a curvature to act as a lens that focuses light energy from light source 202.

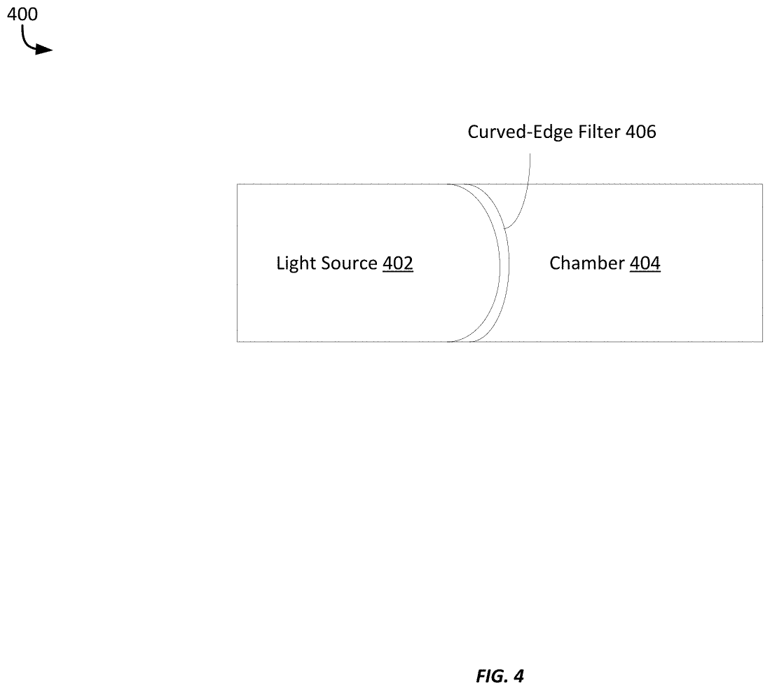

As shown in FIG. 2A, light source 202 is coupled to a filter 204. Filter 204 comprises a filter configured to remove one or more types of light. For example, filter 204 may comprise a filter configured to remove all light that is not within a certain frequency range (e.g., the frequency range associated with violet light or the frequency range associated with infrared or ultraviolet light). Thus, filter 204 may prevent a light source other than light source 202 from activating propellant 208. For example, filter 204 may be tuned to allow only light energy at the same wavelength as output by light source 202 to pass. This may prevent an interfering light source or outside light source from outputting light energy onto propellant 208, and thus prevent unintended ignition of propellant 208. As is described in further detail below with regard to FIG. 4, in some embodiments, filter 204 may comprise a lensed shape to focus light energy received from light source 202.

Filter 204 is configured to filter all light energy passing into chamber 206. Chamber 206 is sealed on its side facing light source 102 and filter 104 and comprises a piston on its opposite side. Chamber 206 is configured to contain propellant 208. For example, chamber 206 may comprise a chamber similar to Chambers 1 and 2 described above with regard to FIGS. 1A and 1B. Chamber 206 may comprise an enclosed shell made of a substantially firm material, e.g., a firm plastic material.

A piston 210 is positioned within chamber 206. Piston 210 is sized to have approximately the same cross-section as chamber 206, and may comprise a gasket or other seal to prevent the contents of the chamber 206 from sliding around the piston 210 or gas pressure generated behind the piston 206 from being dissipated by escaping around the piston 206.

When propellant 208 is activated (e.g., ignited) it generates pressure within the portion of the chamber behind the piston 210, thereby forcing the piston 210 toward the opposite end of the chamber.

Propellant 208 comprises an ignitable substance configured to generate pressure to press piston 210 forward. For example, propellant 208 may comprise a nitrocellulose material, e.g., either paper or cotton based nitrocellulose. Propellant 208 is configured to be ignited when it receives light energy from light source 202.

When the propellant 208 is ignited it releases gasses, increasing the pressure inside chamber 206. This increase in pressure applies pressure to piston 210. This pressure forces piston 210 forward. Piston 210 may press a hollow needle 220 forward into a wearer. Alternatively, piston 210 may inject substance 212 into the wearer via a hollow needle 220. In some embodiments, substance 210 may comprise, e.g., glucagon, epinephrine, insulin, saline solution, or any other injectable solution. Further, in some embodiments, the propellant 208 may be selected based on the type of substance 212. For example, the speed at which the propellant ignites may be selected based in part on the viscosity of substance 212.

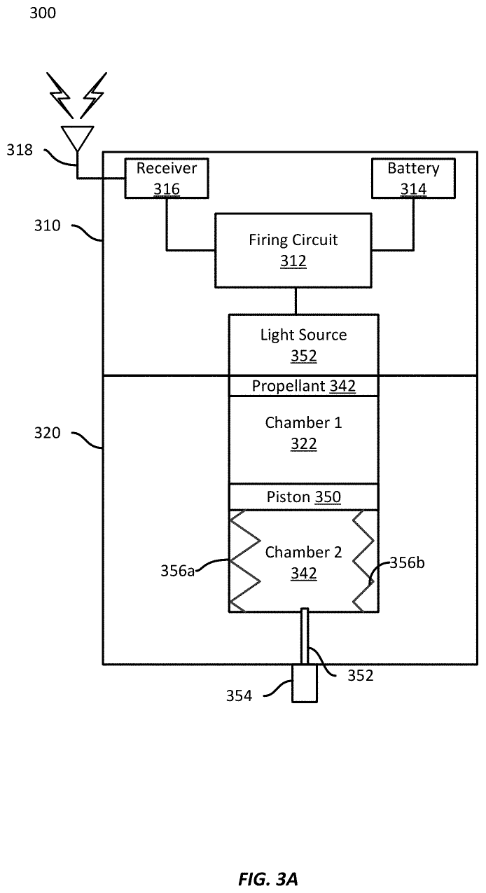

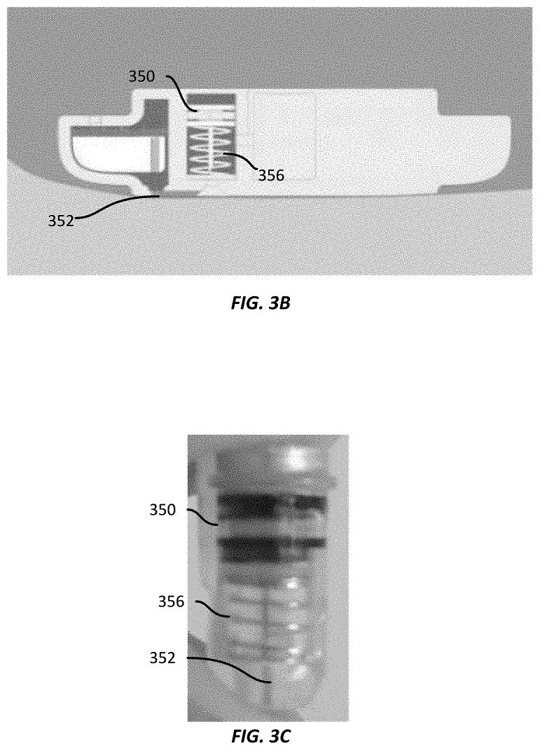

Further, as described in further detail below with regard to FIGS. 3A-3C, in some embodiments, the needle 220 may alternatively be used for blood-extraction.

Turning now to triggering circuitry 240, which comprises a power source 214, control circuit 216, and receiver 218. Power source 214 comprises a power source configured to provide electrical energy to control circuit 216 and light source 202. For example, power source 214 may comprise a battery (e.g., a NiCad, lithium ion, alkaline, dry cell, or other type of battery).

In some embodiments, power source 214 further comprises a switching power supply configured to provide a high voltage pulse to the light source 202. In some embodiments, a switching power supply may enable smaller or more easily worn batteries (e.g., Lithium, CR2032, button, coin, or watch cell) to be used. Further, a switching power supply may provide an additional safety feature in that a battery, by itself, cannot output a charge large enough to cause the light source 202 to ignite the propellant 208.

The control circuit 216 comprises a circuit configured to provide power from power source 214 to light source 202. In some embodiments, control circuit 202 may comprise an electric switch (e.g., a transistor based switch). In other embodiments, control circuit 216 comprises a processor, FPGA, ASIC, or other programmable circuit configured to control light source 202. Further, control circuit 216 is coupled to an antenna 218, which is configured to receive wireless signals. For example, wireless signals received from a wearable sensor (e.g., a continuous glucose monitor) or a handheld device (e.g., a smartphone) and control the light source 202 to activate (e.g., ignite or detonate) propellant 208 based on those wireless signals. For example, in one embodiment a wearable analyte sensor may detect that a condition of the wearer has exceeded a threshold and transmit a signal to control circuit 216 via antenna 218 to ignite propellant 208 to administer the substance 212 to the wearer. In some embodiments, control circuit 216 is electrically coupled to an alert system (e.g., an audible or visual alert system) and further configured to provide a visual or audible warning to the wearer prior to controlling light source 202 to ignite propellant 208.

While this example employs a wireless command to activate the triggering circuit 240, in some examples, the device 200 may instead have a wired connection to another device, e.g., a biosensor, or may have a button or other wearer manipulatable device (“manipulandum”) to activate the control circuit 210. Further, in some embodiments, the wired or wireless signal may be encrypted to protect confidentiality. Further, in some embodiments, status information associated with the device may be stored remotely (e.g., at a remote device or via a remote network such as the cloud) and provided periodically to a health care provider.

Further, in addition to the control circuit 216, other electronic components may be provided, such as battery charging circuitry, power and filtering circuitry, and a microcontroller, e.g., an ASIC defined on a field-programmable gate array (“FPGA”). Still further electronic components may be included to enable various features according to this disclosure.

Turning now to FIG. 2B, FIG. 2B shows another embodiment of an example system for triggering a drug injection device. The embodiment shown in FIG. 2B comprises a triggering circuit 250. As shown in FIG. 2B, triggering circuit 250 comprises a power source 252, control circuit 254, antenna 256, and light source 258. As shown in FIG. 2B, light source 258 may comprise one or more of a lamp or any type of LED (e.g., a white, blue, green, red, laser LED, or infrared LED). Light source 258 is configured to provide light energy to a propellant, causing the propellant to heat and ignite.

Power source 252 comprises a power source configured to provide electrical energy to control circuit 254 and light source 258. For example, power source 252 may comprise a DC power source such as a battery. In some embodiments, power source 252 may further comprises a switching power supply or transistor configured to act as a “charge pump” to provide a higher voltage to light source 258 than would ordinarily be generated by a battery.

As shown in FIG. 2B, control circuit 254 comprises a transistor. When power is provided to the base of the transistor, it allows current to flow from power source 252 to light source 258. In other embodiments, control circuit 254 may comprise a more complex circuit, e.g., a plurality of transistors and/or amplifiers. In still other embodiments, control circuit 254 may comprise a processor, FPGA, ASIC, or other programmable circuit coupled to a memory configured to contain program code to cause the programmable circuit to carry out functions described herein.

Control circuit 254 is electrically coupled to antenna 256. Antenna 256 is configured to receive wireless signals. For example, wireless signals received from a remote device such as a wearable sensor (e.g., a continuous glucose monitor) or a handheld device (e.g., a smartphone). In some embodiments, the remote device may determine that an injectable substance should be provided to the wearer and provide a signal via antenna 256 to cause control circuit 254 to activate light source 258 and thereby ignite a propellant to provide an injectable substance to the wearer.

In one embodiment, a remote sensor may detect that some measurement associated with the wearer has gone beyond a threshold (e.g., the wearer's blood sugar, blood pressure, or blood oxygen content has passed above or below a threshold). The sensor may then transmit a signal to antenna 256 (e.g., via Bluetooth, Bluetooth Low Energy (BLE) WiFi, NFC), this signal causes control circuit 254 to apply current to light source 258. Light energy from light source 258 causes a propellant to ignite, which generates sufficient pressure to inject an injectable substance into the wearer.

Turning now to FIG. 2C, FIG. 2C shows another embodiment of an example system for triggering a drug injection device according to the present disclosure. The embodiment shown in FIG. 2C comprises a receiver 218, control circuit 216, power source 214, propellant 208, and light source 202, which are all similar to corresponding components described above with regard to FIG. 2A. Each of these components may be part of a device similar to that described above with regard to FIGS. 1A-1D or 2A.

The embodiment in FIG. 2C further comprises one or more photovoltaic cells 262, which are light sensitive materials configured to receive light energy and convert that light energy to electrical energy. As shown in FIG. 2C, one or more photovoltaic cells receive light energy from light source 202 and converts that light energy to electrical energy. This electrical energy is then provided to propellant 208 to ignite the propellant. Further, in some embodiments, the electrical energy may be provided to another device, e.g., a resistor or other conductor, that generates heat sufficient to ignite the propellant 208.

FIG. 3A shows an embodiment of an example system for triggering a blood extraction device according to the present disclosure. As can be seen in FIG. 3A, the example device 300 has two portions 310, 320 that are connected, but are separable from each other. The first portion 310 has electronic components within it, which are similar to the electronic components described above with regard to FIGS. 1B, 2A, and 2B, and an antenna 118 to receive wireless signals. The first portion 310 in this example is separable from the second portion 320 to allow for re-use of the electronics, while the second portion can be discarded after it has been used.

The second portion 320 has two chambers that are separated by a piston 350. The second portion further comprises a hollow needle 352 and a needle cap that can be used to drive the needle 352 through the needle guide 354 and into a person's skin. In this example, because the needle 352 is hollow such that blood can be extracted from the wearer through the needle 352 and into chamber 2 (342).

The example device shown in FIG. 3A is designed to be worn flush against a wearer's body, such as on an upper arm or torso. The needle 352, as shown in FIG. 3A, is oriented to extend parallel to the wearer's skin; however, the needle guide 354 defines a curved path that forces the needle 352 to bend toward the wearer's skin at an angle departing from its initial orientation by approximately 30 degrees in this example. Thus, the needle 352, in this example, is formed of flexible materials, such as a nickel-titanium alloy (e.g., Nitinol), to allow the needle 352 to bend at angles of up to 30 degrees (or more) without breaking or obstructing the fluid path through the interior of the needle 352. In addition, the needle 352 in this example is a 22-gauge needle. Such a needle size may provide a diameter suitable for extracting blood from the wearer while having a diameter that causes a tolerable amount of discomfort; however, other suitable needle diameters may be employed.

As discussed above, the system 300 comprises two chambers, chamber 1 (322) and chamber 2 (324), which are separated by a piston 350. Piston 350 is sized to have approximately the same cross-sectional area as the two chambers, thus when piston 350 moves it generates a pressure differential within chamber 1 (322) and chamber 2 (342).

In the embodiment shown in FIG. 1, a propellant 342 is positioned behind piston 350 within chamber 1 (322). In this example, propellant 342 comprises a nitrocellulose material. When propellant 342 is ignited, pressure builds in chamber 1 (322), which forces piston 350 downward. This action causes needle 352 to be pressed forward into a wearer's skin.

Once the needle 352 is pressed forward, retraction mechanisms 356a, 356b apply return pressure to piston 350. This generates a vacuum within chamber 2 (342) to extract blood from the wearer via needle 352 into the chamber 2 (342). In some embodiments, retraction mechanisms 356a, 356b, or an additional retraction mechanism may further retract the needle from the wearer after blood is extracted. This blood then may be tested to measure, e.g., the presence of an analyte such as blood glucose, sodium, oxygen, or some other measure associated with blood.

The first portion 310 includes components to receive a command (or commands) to activate the propellant and extract blood from the wearer. In this example, the first portion 310 includes a firing circuit 312, a battery 314 or other electrical power source or connection, a wireless receiver 316, and an antenna 318. To activate the propellant 342, a command may be received via the antenna 318 and the receiver 316 from a remote device, such as the wearer's smartphone or a biosensor (e.g., a CGM), and is provided to the firing circuit 312. In response to receiving the command, the firing circuit 312 activates the propellant 342 using power supplied by the battery 314.

In this example, the propellant 342 is activated by optical energy output by light source 352. The light source 352 comprises any type of light source, e.g., an LED, and are controlled by firing circuit 112. While this example employs a wireless command to activate the firing circuit 312, in some examples, the device 300 may instead have a wired connection to another device, e.g., a biosensor, or may have a button or other wearer manipulatable device (“manipulandum”) to activate the firing circuit 312.

With respect to description of length, width, and height, the height of the device 300 shown in FIG. 3A refers to how far the device extends above the wearer's skin when worn as described above. The length and width, by contrast, refer to the dimensions of the perimeter of the device 300 shown in FIG. 3A.

FIG. 3B shows another embodiment of an example system for triggering a blood extraction device according to the present disclosure. FIG. 3B shows a drawing of an embodiment of the system described with regard to FIG. 3A.

FIG. 3C shows another embodiment of an example system for triggering a blood extraction device according to the present disclosure. FIG. 3C shows an image of an embodiment of the system described with regard to FIG. 3A.

Turning now to FIG. 4, FIG. 4 shows another embodiment of an example system for triggering a drug injection device. The embodiment shown in FIG. 4 shows an exploded view of the barrier between a light source (e.g., light source 202 shown in FIG. 2A) and a chamber (e.g., chamber 206 shown in FIG. 2A). FIG. 4 shows a light source 402 and chamber 404, which are separated by a curved-edge filter 406. The light source 402 comprises a light source similar to light source 402 described above with regard to FIG. 2A. The chamber 404 comprises a chamber similar to chamber 206 described above with regard to FIG. 2A.

FIG. 4 further shows a curved-edge filter 406 positioned directly between light source 402 and chamber 404. In some embodiments, curved-edge filter 406 may comprise a filter configured to remove all light that is not within a certain frequency range (e.g., the frequency range associated with violet light or the frequency range associated with infrared or ultraviolet light). Thus, curved-edge filter 406 may prevent a light source other than light source 402 from activating a propellant within chamber 404. For example, curved-edge filter 406 may be tuned to allow on light energy at the same wavelength as output by light source 402 to pass. This may prevent an interfering light source or outside light source from outputting light energy onto a propellant within chamber 404.

In some embodiments, the curved edge of curved-edge filter 406 is configured to act as a lens that focuses light received from light source 402. The focal point of the lens may fall substantially on a location in chamber 404 at which a propellant (similar to propellant 208 described above with regard to FIG. 2A) is located. The curved-edge filter 406 may cause a greater amount of light energy to fall on the propellant and thus cause the propellant to ignite more quickly when light source 402 outputs light energy. In some embodiments, rather than a separate component, curved-edge filter 406 may comprise a component of chamber 404.

Further, in some embodiments, light source 402 may comprise a monochromatic light source (e.g., a laser). Monochromatic light may be more easily focused into a very small area, increasing the intensity of light on that small area. In some embodiments, this may lead to faster or more efficient ignition of the propellant.

Illustrative Method for Triggering a Drug Injection Device

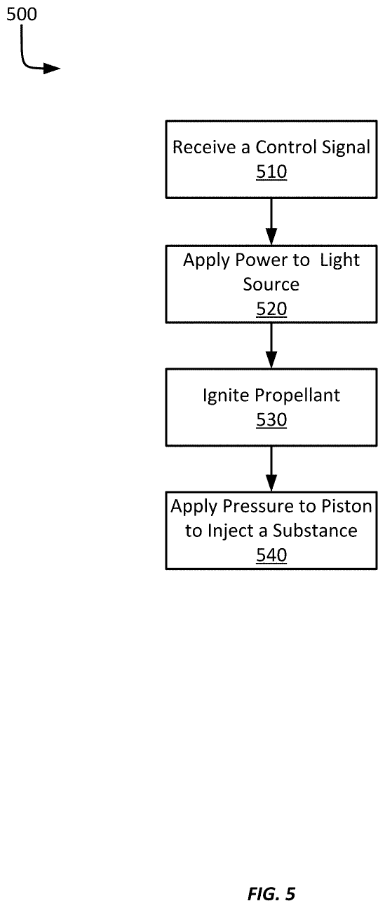

Referring now to FIG. 5, FIG. 5 shows an example method 500 for triggering a drug injection device. In some embodiments, the steps in FIG. 5 may be performed in a different order. Alternatively, in some embodiments, one or more of the steps shown in FIG. 5 may be skipped, or additional steps not shown in FIG. 5 may be performed. The steps below are described with reference to components described above with regard to the device 200 shown in FIG. 2A.

The method 500 begins at step 510 when control circuit 216 receives a control signal. In some embodiments the control signal may be received wirelessly via an antenna 218. In other embodiments, the control signal may be received via a wired connection. In some embodiments the control signal is received from a remote device such as a wearable sensor (e.g., a continuous glucose monitor) or a handheld device (e.g., a smartphone). In some embodiments, the remote device may determine that an injectable substance should be provided to the wearer device, and thus provide a control signal to control circuit 216.

Next at step 520 the control circuit 216 applies power to a light source 202. In some embodiments, control circuit 216 comprise a switch configured to control the flow of power between a power source 214 and a light source 202. For example, in one embodiment, control circuit 216 may comprise an electronic switch that opens when it receives a control signal.

At step 530 the light source 202 ignites a propellant 208. Light source 202 is configured to apply light energy to a propellant 208. This light energy may heat propellant 208 to the point that it ignites. Light source 202 may comprise one or more of a lamp or any type of LED (e.g., a white, blue, green, red, laser LED, or infrared LED). In some embodiments, an end of chamber 206 is curved to focus the light generated by light source 202 onto the propellant 208. Further, in some embodiments, a filter 204 is positioned between light source 208 and propellant and configured to remove all light that is outside of a certain range. Filter 204 may prevent unintentional ignition of the propellant 208. For example, in one embodiment, light source 202 may comprise a violet LED and filter 204 may be configured to filter all light outside of the frequency associated with violet light to prevent an outside light source from igniting propellant 208. In some embodiments, filter 204 provides additional safety by preventing unintended ignition, because filter 204 makes the system 200 more immune to stray sources that could ignite the propellant 208 (e.g., RF fields from cell phones or access control door card readers, etc.).

Then at step 540 pressure is applied to a piston 210, which injects a substance 212 into a wearer. Chamber 206 may be sealed an all but one side, which comprises a piston 208. When propellant 208 ignites it releases gasses that increase the pressure in chamber 206. This increase in pressure forces piston 210 forward, which causes substance 212 to be injected into a wearer (e.g., under the wearer's skin or into the wearer's bloodstream) via a hollow needle 220.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

That which is claimed is:

1. A device for injecting a substance into a patient comprising:
a chamber comprising a propellant;
a light source mechanically coupled to the chamber and positioned to emit light toward the propellant to ignite the propellant; and
a power source electrically coupled to the light source via a control circuit, wherein the control circuit applies power to activate the light source.

2. The device of claim 1, wherein the light source comprises a Light Emitting Diode (LED).

3. The device of claim 2, wherein the chamber comprises a molded end to act as a lens to focus light from the light source.

4. The device of claim 2, further comprising a light filter mechanically coupled between the light source and the chamber.

5. The device of claim 4, wherein the light filter blocks all light outside of a frequency band associated with violet light.

6. The device of claim 1, wherein the propellant comprises nitrocellulose in a cotton-based format or a paper-based format.

7. The device of claim 1, wherein the control circuit comprises an electronic switch controlled by a processor communicatively coupled to a remote device, and wherein the remote device comprises one or more of: a sensor, a smartphone, a smartwatch, a continuous glucose monitor, insulin pump, or a wearable device.

8. The device of claim 7, wherein the sensor comprises one or more of: an analyte sensor, a blood pressure sensor, or an Electrocardiogram (ECG) sensor.

9. The device of claim 8, wherein the analyte sensor comprises one or more of: a continuous glucose monitor (CGM) or a blood oxygen sensor.

10. The device of claim 1, wherein the ignited propellant generates a pressure on a piston to inject a substance into the patient, wherein the substance comprises one or more of: insulin, epinephrine, glucagon, or a glucagon activation solution.

11. A method for injecting a substance into a patient comprising:
receiving a control signal;
controlling a power supply to apply power to a light source mechanically coupled to a chamber, wherein energy from the light source ignites a propellant within the chamber; and
applying pressure to a piston within the chamber to inject a substance into a patient.

12. The method of claim 11, wherein the light source comprises a Light Emitting Diode (LED).

13. The method of claim 12, wherein the chamber comprises a molded end to act as a lens to focus light from the light source.

14. The method of claim 12, wherein the chamber comprises a light filter to filter light received from the light source.

15. The method of claim 14, wherein the light filter blocks all light outside of a frequency band associated with violet light.

16. The method of claim 11, wherein the propellant comprises nitrocellulose in one or more of a cotton-based format or a paper-based format.

17. The method of claim 11, wherein the control signal is received from a processor communicatively coupled to a remote device, and wherein the remote device comprises one or more of: a sensor, a smartphone, a smartwatch, or a wearable device.

18. The method of claim 17, wherein the sensor comprises one or more of: an analyte sensor, blood pressure sensor, or an Electrocardiogram (ECG) sensor.

19. The method of claim 18, wherein the analyte sensor comprises one or more of: a continuous glucose monitor (CGM) or a blood oxygen sensor.

20. The method of claim 11, wherein the substance comprises one or more of: insulin epinephrine, glucagon or a glucagon activation solution.
